(19) United States
(12) Patent Application Publication
(10) Pub. No.: US 2002/0172730 A1
(43) Pub. Date: Nov. 21, 2002

(54) Orals dosage noni formulations

(75) Inventors: Trevor Page, Salt Lake City, UT (US); Darald Donnell, Long Grove, IL (US)

Correspondence Address: THORPE NORTH WESTERN, 8180 SOUTH 700 EAST, SUITE 200, P.O. BOX 1219, SANDY, UT, 84070, US

(73) Assignee: Lifesmart Nutrition, Inc.

(21) Appl. No.: 09/834,178

(22) Filed: Apr. 12, 2001

(51) Int. Cl. A61K 35/78

(52) U.S. Cl. 424/769; 424/765

(57) ABSTRACT

The present invention provides a Noni extract formulation which reduces or eliminates the strong disagreeable Noni taste, while simultaneously increasing the efficiency of a Noni extract dose. In one aspect, a Noni extract is uniformly dispersed in a caramel composition having an insulin response enhancing amount of sugar. In another aspect, the caramel composition may include an effective amount of an invert sugar.

### THE FIELD OF THE INVENTION

The present invention relates generally to a composition and method for administering Noni plant extracts. More particularly, it concerns a Noni plant extract formulation which eliminates unpleasant Noni taste and enhances Noni metabolism and absorption.

### BACKGROUND OF THE INVENTION

Morinda citrifolia, a small evergreen tree commonly referred to as “Indian Mulberry,” is indigenous to various south pacific costal regions and islands, and has been used for centuries in traditional folk medicine to treat a variety of ailments. Today, known best as the “Noni plant,” many people believe that Morinda citrifolia extract is useful in treating diabetes, cancer, ulcers, heart trouble, high blood pressure, kidney and bladder disorders, as well as a myriad of other physical conditions.

Noni plant extract is most often prepared for oral dosage delivery as a liquid infusion or tincture of the Noni plant fruit. Unfortunately, fresh Noni fruit and liquid preparations thereof generally have a strong disagreeable taste and odor. Additionally, because liquid oral formulations are often bulky, may require refrigeration, and are messy if spilled, they are inconvenient for multiple daily dose regimens. This is especially true for individuals who lead an active lifestyle and may travel throughout the day.

In order to alleviate the problems associated with liquid oral dosages of Noni plant extract, a variety of powdered forms have become available which may be delivered as an oral dosage tablet or capsule. Such formulations are thought to eliminate the strong disagreeable Noni plant taste and smell. However, it has been found that the Noni taste may still be experienced to an unpleasant degree as a residual taste after oral consumption of tablets or capsules.

Additionally, metabolism and absorption of orally administered Noni extract by body tissues has been found to be less than optimal. Particularly, as with many other substances, cellular metabolism and absorption of Noni is enhanced in the presence of insulin. Unfortunately, Noni's therapeutically active ingredients are very susceptible to degradation by the digestive forces of the upper gastrointestinal tract. Therefore, administration of a Noni dose in close proximity to a nourishment event that is sufficient to significantly raise insulin levels actually reduces Noni dosage efficacy.

As a result, research and development efforts continue to pursue Noni fruit extract dosage formulations which are easily consumable, portable, and that maximize metabolism and absorption by the body.

### SUMMARY OF THE INVENTION

Accordingly, the present invention provides a Noni extract formulation which includes a caramel or taffy base having an effective amount of a Noni extract dispersed therein. In one aspect, the amount of Noni extract may be from about 1% to about 25% w/w of the formulation. In another aspect, the amount of Noni extract may be from about 5% to about 15% w/w of the formulation.

A wide variety of Noni plant types may be utilized in connection with the present invention for producing an acceptable Noni extract. In one aspect, the source of the Noni extract may be a member of the group consisting of Tahitian Noni plants, Hawaiian Noni plants, Samoan Noni plants, and mixtures thereof. In another aspect, the Noni extract may be obtained from a Samoan Noni plant.

Numerous active agents in Noni extract have been indicated as causing the positive health benefits imparted. One such agent is polysaccharide. In one aspect, the Noni extract used in the present invention may include a therapeutically effective amount of a polysaccharide. In another aspect, the amount of polysaccharide may be from about 2% to about 5% w/w of the Noni extract. In yet another aspect, the amount of polysaccharide may be about 3% w/w of the Noni extract.

An additional active agent which is reputed to play a role in imparting positive health benefits is proxeronine and its activating enzyme proxeroninase. In one aspect, the Noni extract used in the present formulation may include a therapeutically effective amount of proxeronine. In another aspect, the amount of proxeronine may be from about 0.1% to about 50% w/w of the Noni extract. In yet another aspect, the amount of proxeronine may be about 5% w/w of the Noni extract.

The amount of sugar in the caramel or taffy base of the present invention may be an amount sufficient to mask or reduce the objectionable Noni extract taste, and may also be sufficient to rapidly enhance insulin levels. The total sugar in the caramel base may include an effective amount of an invert sugar. In one aspect, the amount of invert sugar may be from about 1% to about 20% w/w of the formulation. In another aspect the amount of invert sugar may be from about 3% to 15% w/w of the formulation.

A variety of invert sugar types may be utilized with the present invention to provide a heightened sweetening effect. In one aspect, the invert sugar may be a mixture of dextrose (i.e. D-glucose) and fructose. In another aspect, the dextrose and the fructose may each be present in an amount of about 50% w/w of the invert sugar. In yet another aspect, the invert sugar may be provided by rice syrup and include a mixture of glucose and maltose. In a further aspect, the amount of rice syrup may be from about 15% to about 40% w/w of the formulation.

The present invention additionally provides a Noni extract formulation which includes a caramel or taffy base having an insulin enhancing amount of sugar and invert sugar, and a therapeutically effective amount of a Noni extract, wherein said invert sugar is present in an amount sufficient to reduce a disagreeable taste imparted by the Noni extract, and the formulation has a total dosage size or amount that is insufficient increase upper gastro intestinal tract digestive activity to a level or degree which substantially inactivates one or more active agents, or a substantial portion thereof, in the Noni extract.

The present invention also encompasses a method for making a Noni extract. In one aspect, a method of making a Noni extract formulation comprising the steps of: a) preparing a caramel or taffy base containing an effective amount of an invert sugar in a conventional manner in a heated liquid caramel phase form; b) partially cooling said heated liquid caramel phase to a temperature at which Noni extract is stable; c) adding a desired amount of Noni extract to said partially cooled liquid caramel phase; d) agitating said partially cooled liquid caramel phase until the Noni extract is substantially uniformly dispersed therein; and e) further cooling said partially cooled liquid caramel phase to a solid thereby resulting in said Noni extract formulation.

In one aspect, the temperature of said partially cooled liquid caramel phase may be between about 160° F. to about 220° F. at the time said Noni extract is added. In another aspect, the temperature is from about 180° F. to 200° F.

In one aspect, the amount of Noni extract added may be from about 0.1% to 24% w/w of the Noni formulation. In another aspect, the amount of Noni extract may be from about 9% to 13% w/w of the Noni formulation.

In one aspect, the invert sugar is provided by an effective amount of rice syrup. Further, the present method may additional include the step of dividing the Noni extract formulation into individual serving size portions.

Another method included in the present invention is a method of increasing the efficacy of a Noni extract dose. Such a method may include the steps of: a) distributing an amount of Noni extract into a caramel composition containing an insulin enhancing amount of sugar and an invert sugar; and b) orally administering the composition.

In one aspect, the amount of invert sugar may be from about 1% to about 20% w/w of the formulation. In another aspect, the amount of invert sugar may be about 3% to 15% w/w of the formulation. In a further aspect, the invert sugar includes a mixture of dextrose and fructose. In yet another aspect, the dextrose and the fructose may each be present in an amount of about 50% w/w of the invert sugar. In an additional aspect, the invert sugar may be provided by rice syrup and includes a mixture of glucose and maltose. In yet another aspect, the amount of rice syrup is from about 15% to about 40% w/w of the formulation.

The method of enhancing the efficacy of a Noni extract dose may additionally include the step of administering the composition at a time when digestive juices in the upper gastrointestinal tract are at a minimum. Additionally, Noni formulation may be administered in a total amount that is insufficient to increase upper gastro intestinal tract digestive action to a level which substantially inactivates one or more active agents, or a substantial portion thereof contained in the Noni extract.

In addition to the above-recited methods, the present invention includes a method of reducing or eliminating an objectionable taste caused by Noni extract. Such a method may include the steps of: a) distributing the Noni extract into a caramel composition containing a sufficient amount of invert sugar to reduce or eliminate any disagreeable taste caused by the Noni extract.

In one aspect, the amount of invert sugar may be from about 1% to about 20% w/w of the formulation. In another aspect, the amount of invert sugar may be from about 3% to 15% w/w of the formulation. In a further aspect, the invert sugar may include a mixture of dextrose and fructose. In yet another aspect, the dextrose and the fructose are each present in an amount of about 50% w/w of the invert sugar. In an additional aspect, the invert sugar may be provided by rice syrup and includes a mixture of glucose and maltose. In another aspect, the amount of rice syrup is from about 15% to about 40% w/w of the formulation.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION

Definitions

Before the present oral delivery Noni formulations are disclosed and described, it is to be understood that the present invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used.

The singular forms “a,” and, “the” include plural referents unless the context clearly dictates otherwise. Thus, for example, reference to a caramel containing “a Noni component” includes one or more Noni components, reference to “a sugar” includes reference to one or more sugars, and reference to “the flavorant” includes reference to one or more flavorants.

In describing and claiming the present invention, the following terminology will be used in accordance with the definitions set forth below.

As used herein, “Noni,” “Noni fruit,” “Noni plant,” “Noni agent,” and “Noni extract,” refer to an extract made from the fruit of all strains and hybrids of the plant Morinda citrifolia, or of plants significantly related to it, grown anywhere in the world including blends, mixtures, and combinations of such strains and relatives.

The terms “formulation” and “composition” may be used interchangeably herein.

As used herein, a “sugar” refers to any type of simple carbohydrate, such as a mono or disaccharide, or a combination thereof, either naturally obtained, refined from a natural source, or artificially produced, which may act as a suitable sweetener in a caramel composition.

As used herein, “inactivate” refers to a reduction, or substantial reduction in therapeutic action which would be imparted by an active agent when administered to the body.

As used herein, “invert sugar” refers to a combination of two or more sugars, either naturally obtained, refined from a natural source, or artificially produced, that produces a greater sweetness than a single type of sugar. By way of example without limitation, an invert sugar may include a mixture of fructose and D-glucose in substantially equal parts. One example of a naturally obtained invert sugar is rice syrup. Rice syrup is generally obtained by culturing rice with certain enzymes to break down starches, straining off the liquid, and cooking the remaining portion until a desired consistency is reached. The resultant product contains a mixture of soluble complex carbohydrates, maltose, and glucose. In such a case, the combination of maltose and glucose act much like the more traditional combination of fructose and D-glucose.

As used herein, “chew” and “chew base” may be used interchangeably and refer to either a caramel or taffy base.

As used herein, “caramel,” and “caramel base,” may be used interchangeably, and refer to a smooth, chewy composition made with sugar, butter or other fats, cream or milk or milk solids, and flavoring. Such ingredients may be unaltered natural products, natural products which are processed or refined, or may be fully synthesized products.

As used herein, “taffy,” or “taffy base” refers to a chew candy or confection which is made with various types of sugars, including but not limited to simple sugars, invert sugars, brown sugars, and molasses, which is boiled until very thick and then pulled until it is glossy and holds its shape.

As used herein, “artificial sweetener” refers to a sweetening agent which does not provide a substantial amount of calories when consumed, as compared to the calories provided by an amount of sugar required to impart an equivalent sweetening effect. A variety of artificial sweeteners are known to those skilled in the art, including without limitation, saccharin, aspartame, sucralose, etc.

As used herein, an “effective amount,” and “sufficient amount” may be used interchangeably and refer to an amount of an ingredient which, when included in a chew composition, is sufficient to achieve an intended compositional or physiological effect. For example, a “sufficient amount” of invert sugar would be the minimum amount needed to reduce or eliminate an off or disagreeable taste caused by an amount of Noni extract. Further, a “therapeutically effective amount” refers to an amount of a Noni extract which is sufficient to achieve a desired physiological effect. The determination of an effective amount is well within the ordinary skill in the art of pharmaceutical, neutraceutical, herbaceutical, cosmetic, and medical sciences. See, for example, Meiner and Tonascia, “Clinical Trials: Design, Conduct, and Analysis,” Monographs in Epidemiology and Biostatistics, Vol. 8 (1986), incorporated by reference in its entirety.

As used herein, “an insulin enhancing amount,” or “an insulin level enhancing amount” of a substance refers to an amount of sugar or other nutritional agent that is sufficient to produce or raise the amount of insulin in the blood to a level which increases the metabolism and absorption of Noni fruit extract, or the active agents contained therein, to a rate or amount which is greater than at a lower insulin level. Various methods for measuring and determining various insulin levels and their effect on the metabolism and absorption of various nutritional components are well known to those in the art.

As used herein, “active agent” refers to an agent contained in a Noni extract which imparts, or is capable of imparting or inducing a measurable physiological effect when administered to the body. Examples of active agents include proxeronine, proxeroninase, polysaccharides, terpenes, alkaloids, vitamins, minerals, etc.

As used herein, “polysaccharide” refers to a compound containing a combination of nine or more monosaccharides which are linked together by glycosidic bonds.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a concentration range of “about 0.1% w/w to about 25% w/w” should be interpreted to include not only the explicitly recited concentration of about 0.1% to about 25% w/w, but also include individual concentrations and the sub-ranges within the indicated range. Thus, included in this numerical range are individual concentrations such as 2% w/w, 5% w/w, and 6% w/w, and sub-ranges such as from 1% w/w to 3% w/w, from 2% w/w to 6% w/w, from 8% w/w to 18% w/w, from 5% w/w to 20% w/w, etc. The same principle applies to ranges reciting only one numerical value.

Similarly, a range recited as “less than about 5.8% w/w” should be interpreted to include all of the values and ranges as elaborated above for the range of “from about 0.1% w/w to about 25% w/w.” Furthermore, such an interpretation should apply regardless of the breadth of the range or the characteristics being described.

Invention

The present invention is drawn to an oral dosage Noni extract formulation which includes a therapeutically effective amount of Noni extract contained in a chewy confection type vehicle such as a caramel or taffy. Such a formulation provides significantly improved taste, convenience, and efficiency aspects over conventional liquid, tablet, or capsule formulations.

Single Noni formulation dosage sizes typically range from about 4 to about 12 grams total weight and are individually wrapped for convenient transport and administration. The amount of Noni extract contained in a chew of this size may be from about 0.5 to 1.5 mg. In one aspect, the total weight for a single dosage may be about 8.6 grams, and the amount of Noni extract contained therein may be about 500 mg.

The Noni component of the present invention is generally included as a powder, and may be obtained by any process of active ingredient extraction known to those skilled in the art. By way of example, without limitation, extraction techniques, such as infusion, tincture, etc. followed by removal of the liquid portion and concentration of the extract may be used. One such method for producing a powdered Noni extract is described in U.S. Pat. No. 5,288,491 which is incorporated herein by reference in its entirety.

The amount of Noni component contained in the formulation may be varied according the knowledge of one skilled in the art in order to achieve a particularly desired result. However, the Noni content will be generally from about 1% w/w to about 25% w/w of the formulation. In one aspect, the amount may be from about 5% w/w to about 15% w/w of the formulation. In another aspect, the amount may be about 6% w/w of the formulation.

A variety of beneficial active ingredients are contained within Noni extract. By way of example, without limitation, beneficial ingredients include vitamins, minerals, enzymes, terpenes, proteins, polysaccharides, and alkaloids. Of these ingredients, significant health effects such as anti-cancer and blood pressure lowering effects have been generally attributed to the alkaloids and polysaccharides.

The beneficial alkaloid ingredients have been identified as proxeronine and an enzyme known as proxeroninase. The action of proxeronine and proxeroninase, also known as proxeronase, in forming an alkaloid known as xeronine are more fully disclosed and described in U.S. Pat. Nos. 4,409,144, and 4,543,212, each of which are incorporated herein by reference in their entirety. Further, the positive health imparting properties of xeronine, as well as information on the formation thereof is described by Dr. Neil Soloman in his book entitled The Noni Phenomenon: Discover the Powerful Tropical Healer that Fights Cancer, Lowers High Blood Pressure, and Relieves Chronic Pain (1999), which is incorporated herein by reference.

In one aspect, the Noni extract contained in the present formulation may have a proxeronine content of from about 0.01% w/w to about 95% w/w of the total Noni extract. In another aspect, the proxeronine content may be from about 5% w/w to about 50% w/w of the total Noni extract. The total amount of proxeronine contained in a specific amount of the present Noni formulation may be readily determined by those ordinarily skilled in the art. Simply, using the concentration of proxeronine in the Noni extract, and the amount of extract in the formulation, the total proxeronine amount in the formulation may be calculated.

The amount of the proxeroninase in the Noni extract will generally have an activity sufficient to activate at least a portion of the proxeronine contained therein under proper conditions. In one aspect, the activity of proxeroninase may be sufficient to activate at least 50% of the proxeronine. In another aspect, the activity of proxeroninase may be sufficient to activate at least 75% of the proxeronine. In yet another aspect, the activity of the proxeroninase may be sufficient to activate 100% of the proxeronine.

Various sources attribute many of the positive health benefits of Noni to its polysaccharides content, including water soluble polysaccharides. For example, Hirazumi et al., An immunomodulatory polysaccharide-rich substance from the fruit juice of citrifolia (noni) with anti-tumor activity, Phytother Res. 1999 Aug: 13(5):380-7, which is incorporated herein by reference, reports that the anti-cancer effects of Noni are attributed to the polysaccharide content.

In one aspect, the Noni extract utilized in the present invention may have a polysaccharide content of from about 0.01% w/w to about 25% w/w of the total Noni extract. In one aspect, the polysaccharide content may be from about 1% w/w to about 10% w/w of the total Noni extract. In yet another aspect, the polysaccharide content may be from about 2% w/w to about 5% w/w of the total Noni extract. The total amount of polysaccharides contained in a specific amount of the present Noni formulation may be readily calculated by one skilled in the art using the amount of polysaccharide in the Noni extract and the amount of Noni extract in the formulation.

The capability of invert sugar to combat the disagreeable taste of Noni fruit extract is due to its particular nature. Invert sugar is generally a combination of the simple sugars dextrose (D-glucose) and fructose which provides a sweetness exceeding that of a single type of sugar. In one aspect, invert sugar may be a product of the action of the enzyme invertase on sucrose to form a mixture of levulose fructose) and D-glucose (dextrose). However, invert sugar, as defined herein, may be any combination of simple sugars which imparts a heightened sweetness. In one aspect, the invert sugar used may be that containing an equal parts mixture of D-glucose and fructose. In another aspect, the invert sugar used may be a combination of maltose and glucose.

The timing of the sweetening effect of each of the invert sugar components is complimentary. This time variation in part explains the increased sweetness and reduction of objectionable taste. Particularly, the glucose, and maltose or fructose, as simple sugars, provide an initial burst of sweetness as the invert sugar enters the mouth. This quick sweetening masks the initial distaste of Noni extract. The sucrose and corn syrup solids used in making the chew base, being either a disaccharide or starch hydrolysis product, provide a sustained sweetening power during chewing. Further, the maltose or fructose, while involved in the above two mentioned states, are also believed to provide a lingering sweetness which masks the objectionable Noni extract aftertaste or residual taste.

Notably, a variety of artificial sweeteners may also be used to mask the objectionable Noni extract taste. In one aspect, one or more artificial sweeteners may be used in addition to the sugars present in the chew formulation. In another aspect, one or more artificial sweeteners may take the place of a portion of the sugars present in the chew formulation.

In addition to improving the taste and convenience of a Noni fruit dose, the chew vehicle of the present oral delivery formulation also improves the overall dosage efficacy. As noted above, many of the beneficial Noni agents, such as proxeronine and proxeronase are very susceptible to degradation by the digestive forces of the upper gastrointestinal tract. Therefore, it is recommended that Noni be taken on an empty stomach. However, when taken on an empty stomach, a portion of a Noni fruit dose may escape metabolism and absorption by the body tissues due to low blood insulin levels.

As such, the chew vehicle of the present invention is a particularly well suited vehicle for administering Noni on an empty stomach because of the total amount of combined sugars which are present. Particularly, the total amount of combined sugars in the chew is sufficient to raise insulin levels to a point which enhances Noni agent metabolism and absorption by the cells. Further, the total dosage size of the Noni chew formulation is relatively small and does not by itself facilitate significant production of digestive substances in the upper gastrointestinal tract.

Thus, because of its small size, the present Noni formulation prevents loss of Noni extract due to digestion in the upper gastrointestinal tract. Further, because of its high sugars content, the present Noni formulation enhances Noni extract metabolism and absorption in the tissues and organs. As such, the combination of high sugar content and small total administration volume allow the Noni formulation of the present invention to maximizes the efficacy of a Noni dose.

The caramel or taffy composition of the present invention may be a preparation of any combination of ingredients which is known to those ordinarily skilled in the art of making caramel, taffy, or other confections, and is not limited except by a requirement to contain an effective amount of Noni extract.

While no limitation on the form of Noni extract used in the present invention is made, in one aspect, the Noni extract may be a powder. In another aspect, the Noni extract may be a liquid. Further, in one aspect, the Noni extract may be obtained from the fruit of a Tahitian Noni plant. In another aspect, the Noni extract may be obtained from the fruit of a Hawaiian Noni plant. In yet another aspect, the Noni extract may be obtained from the fruit of an Asian Noni plant. In yet another aspect, the Noni extract may be obtained from the fruit of a Samoan Noni plant. In a further aspect, the Noni extract may be obtained from a mixture of any of the above sources.

In addition to the Noni extract active ingredient, other active ingredients may be included in the formulation of the present invention which impart a positive health benefit. As will be recognized by those skilled in the art, a wide variety of positive health benefit imparting ingredients may be selected from herbal and botanical extracts, as well as medicinal compounds and be added as desired in order to achieve a specific therapeutic result. Such additions may be made by the skilled artesian without undue experimentation.

Generally, herbal and botanical extracts are made from all kinds of herb and botanic sources and formulated based on their therapeutic function. For example, anti-flu, bone/joint, brain function, cardiovascular, circulatory, diet, depression, digestion, energy, eye vision, general health, immune system, liver, men's health respiratory, rest, urinary tract, women's health, etc. In one aspect, herbal and botanical extracts for inclusion in the present formulation can be selected from, but not limited to, Ginseng, Ginko Biloba, Dong Quai, Hawthorn berry, St. John's Wort, Saw Palmetto, Kava Kava, Rose Hips, Echinacea, Licorice Root, Grape seed, Chammomile, Sea Buckthorn, Aloe Vera, Cinnamon Bark, Cordyceps, Ho Shou Wu, Dandelion, Gynostemma, mushroom, Notginseng, Dan Shen, and mixtures thereof may be included.

In one aspect, vitamins either water soluble or oil soluble may be added. Water soluble vitamins specifically contemplated by the present invention include, but are not limited to: vitamin B₁, B₂, B₃, B₅, B₆, B₁₂, B₁₃, B₁₅, B₁₇, biotin, choline, folic acid, inositol, para-aminobenzoic acid (PABA), vitamin C, and vitamin P. Additionally, oil soluble vitamins include, but are not limited to: vitamin A, vitamin D, vitamin E, and vitamin K.

Other health imparting substances which may be combined with the desired Noni extract in the formulation of the present invention include amino acids, ionic minerals, and naturally occurring anti-oxidants. The amino acids contemplated include: alanine, arginine, carnitine, gamma-aminobutyric acid (GABA), glutamine, glycine, histidine, lysine, methionine, N-acetyl cysteine, ornithine, phenylalanine, taurine, tyrosine, and valine, but are not limited thereto. Additionally, the ionic minerals contemplated by the present invention for inclusion in an embodiment of the formulation include both anions and cations. Finally, the naturally occurring anti-oxidants contemplated for the formulation of the present invention include: grape seed, beta-carotene, and co-enzyme Q-10, but are not limited thereto.

In one aspect, the amount of invert sugar contained in the prepared chew composition of the present invention may be from about 1% to about 20% w/w of the chew composition. In another aspect, the amount of invert sugar may be from about 3% w/w to 15% w/w of the chew composition. In yet another aspect, the amount of invert sugar may be from about 5% w/w to about 10% w/w of the chew composition. These amounts of invert sugar are in addition to the amount of table sugar (sucrose) or corn syrup solids required by the particular caramel or taffy recipe employed.

As defined above, a basic caramel formulation also contains butter or other fats, and either cream, milk, or milk products. Further, a basic taffy formulation may also contain molasses. The exact types and amounts of each of these ingredients may vary depending on the desired characteristics of the final product. Such exact amounts and types may be readily determined by one ordinarily skilled in the art.

Other ingredients known to the applicant as useful for making a Noni extract containing chew include but are not limited to: water, corn syrup, hydro soy oil, emulsifiers, lecithin, whey solids, sweetened condensed skim milk, flavorants, and vanillin.

In one aspect, the chew base, which is used as the vehicle for containing the Noni extract of the present invention may be partially or entirely made utilizing natural ingredients. Natural invert sugar sources such as rice syrup and sugar sources such as evaporated cane juice (turbinado sugar) may be used as one or more sweetening ingredients. Sweetened and condensed whole or skim milk and whey may be used as milk product ingredients. Coconut oil and mono and diglycerides from vegetable or other natural sources may be used as oil and fat ingredients. Further ingredients which may be used include without limitation Soya lecithin, and natural flavorings, including chocolate and vanilla.

Those of ordinary skill in the art will recognize that the amount of each of the above-recited natural ingredients may be varied in order to achieve a particularly desired result. However, in one aspect, the amount of rice syrup may be from about 10% w/w to about 40% w/w of the formulation. In another aspect, the amount of rice syrup may be about 36% w/w of the formulation. Of particular note is that in general, rice syrup is approximately 48% maltose and glucose and 52% complex carbohydrates. As such, the range of effective invert sugar component provided by the rice syrup may be from about 5% w/w to about 20% w/w.

In one aspect, the amount of turbinado sugar may be from about 15% w/w to about 20% w/w of the formulation. In another aspect, the amount may be about 18% w/w of the formulation.

In one aspect, the amount of sweetened and condensed milk may be from about 13% w/w to about 18% w/w of the formulation. In another aspect, the amount may be about 14% w/w.

In one aspect, the amount of whey may be from about 10% w/w to about 17% w/w of the formulation. In another aspect, the amount may be about 13% w/w.

In one aspect, the amount of coconut oil may be from about 1% w/w to about 5% w/w of the formulation. In another aspect, the amount may be about 2%.

In one aspect, the amount of mono and diglycerides may be from about 0.25% w/w to about 2% w/w of the formulation. In another aspect, the amount may be about 0.5% w/w.

In one aspect, the amount of Soya lecithin may be from about 0.05% w/w to about 0.2% w/w of the formulation. In another aspect, the amount may be about 0.1% w/w.

In one aspect amount of chocolate flavor may be from about 4% w/w to about 8% w/w of the formulation. In another aspect, the amount may be about 6% w/w.

In one aspect, the amount of vanilla flavor may be from about 0.1% w/w to about 0.4% w/w of the formulation. In another aspect, the amount may be about 0.2% w/w.

A method for making the Noni formulation of the present invention encompasses all methods for making caramel or taffy which are known to those ordinarily skilled in the art thereof, and is unlimited, other than to the conditions under which the Noni may be added. Particularly, the Noni must not be exposed to conditions which will cause it to become unfit for its intended purpose by changing forms, decomposition of active ingredients, etc. To this end, some restriction may be applied to the time of Noni addition, and the temperature to which it is subjected.

Therefore, in a one aspect of the invention, the Noni extract is uniformly distributed throughout the chew composition, and is added to a heated chew composition after the composition has been cooled to a temperature at which Noni active ingredients will not degrade. In another aspect, in order to achieve uniform distribution, and ensure Noni extract stability, the temperature will be from about 160° F. to about 220° F., and most preferably, the temperature will be about 180° F. to about 200° F.

In order to achieve uniform distribution of the Noni extract, the chew composition must be sufficiently agitated after adding the creatine. In one aspect, the chew composition is continuously cooled and agitated after the addition of the Noni extract until the composition is sufficiently solid that agitation is not practical. At this point the chew is ready to be divided into individual pieces for packaging. When a taffy base is used, the composition may be pulled after cooling until the desired consistency is reached, prior to division for individual packaging.

Because of the heating and stirring process under which most caramel or taffy compositions are prepared, the amount of ingredient added during processing will vary somewhat from the amount retained in the finally prepared chew composition. This is mostly due to the evaporation of water out of the various components which yields a final composition having a greater percentage of some ingredients which are unaffected by the removal of water. Therefore, a desired Noni extract amount in the prepared chew composition as enumerated above, Noni is added during processing in an amount of about 0.1% to about 24% w/w of the chew composition. In one aspect, the Noni extract is added during processing in an amount of about 4% to about 14% of the chew formulation. In another aspect, the Noni extract is added during processing in an amount of about 5% w/w of the chew formulation.

Additionally, in order to achieve the desired amount of invert sugar enumerated above, invert sugar is added during processing in an amount of about 1% to about 9% w/w of the chew composition. In one aspect, the invert sugar amount added during processing is about 5% w/w of the caramel composition.

The flavors of the final chew composition are unlimited. Any desired flavor may be imparted, as long as attaining the flavor would not render any essential ingredient unfit for its intended purpose. Flavors particularly preferred include but are not limited to: chocolate, strawberry, raspberry, orange, lemon, grape, apple, coffee, and toffee.

The example provided below is illustrative of only one embodiment of making a Noni extract containing chew of the present invention. While the processing conditions and ingredients may be preferred, no limitation thereto is to be inferred.

### EXAMPLE

To the pot of a standard sized gas fired Savage cooker with agitation, was added a blend of 5.12 lbs. of sugar, 14.85 lbs. of corn syrup (43 DE), and 2.51 pounds of water. Agitation was begun at about 100 rpm, and heating of the mixture was commenced. During the heating and agitation, 3.65 lbs. of hyrdo soy oil(98F), 0.08 lbs. of lecithin, and 0.34 lbs. of an emulsifier were weighed into the pot. The temperature was increased during the addition of the ingredients until the temperature of the mixture was approximately 230° F.

Approximately 2.81 lbs. of whey solids were dissolved in about 8 lbs. water, and then 2.25 lbs. of invert sugar (levulose and D-glucose), and 6.16 lbs. of sweetened condensed skim milk were added to the whey and water to form a milk mixture. The milk mixture was added to the pot and heating continued until the combined mixture reached a temperature of approximately 235° F.

The mixture contained in the pot was cooled to 232° F. while stirring continued, and 1.40 lbs. of cocoa liquor , 0.11 lbs. of vanillin, and 0.07 lbs. of butter flavoring were added. Mixing was continued, and the composition temperature was allowed to cool to about 200° F. Upon reaching the temperature of about 200° F., 4.70 lbs. of Noni extract was added to the caramel composition. Mixing was continued, and the composition allowed to cool to a temperature of about 180° F.

Once a temperature of about 180° F. was reached, the caramel composition was removed from the pot and transferred to a cooling table. The composition was allowed to rest upon the cooling table until it reached a temperature of about 91° F., at which time the composition was cut and wrapped into individual pieces.

The above described process yielded a Noni extract containing caramel composition having the following components in the amounts specified:

| | % Amount of | | % Amount of |
|---|---|---|---|
| Ingredient | Composition | Ingredient | Composition |
| Water | 10 | Whey Solids | 6.93 |
| Sucrose | 12.76 | Sweetened | 10.78 |
| | | Cond. Milk | |
| Corn Syrup | 29.76 | Chocolate | 3.42 |
| | | Flavor | |
| Hydro Soy Oil | 10.78 | Vanillin | 0.27 |
| Emulsifier | 0.20 | Butter Flavor | 0.16 |
| Lecithin | 0.20 | Noni Extract | 11.72 |
| Invert Sugar | 4.04 | | |

The Noni extract formulation having the components enumerated above showed excellent flavor, texture, and dissolution qualities in the mouth.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A Noni extract formulation comprising a chew base having a therapeutically effective amount of a Noni extract dispersed therein.

2. The Noni formulation of claim 1, wherein the amount of Noni extract is from about 1% to about 25% w/w of the formulation.

3. The Noni formulation of claim 2, wherein the amount of Noni extract is from about 5% to about 15% w/w of the formulation.

4. The Noni formulation of claim 1, wherein the Noni extract is obtained from a member of the group consisting of Tahitian Noni plants, Hawaiian Noni plants, Samoan Noni plants, and mixtures thereof.

5. The Noni formulation of claim 1, wherein the Noni extract is obtained from a Samoan Noni plant.

6. The Noni formulation of claim 1, wherein the Noni extract includes a therapeutically effective amount of a polysaccharide.

7. The Noni formulation of claim 6, wherein the amount of polysaccharide is from about 2% to about 5% w/w of the Noni extract.

8. The Noni formulation of claim 6, wherein the amount of polysaccharide is about 3% w/w of the Noni extract.

9. The Noni formulation of claim 1, wherein Noni extract includes a therapeutically effective amount of proxeronine.

10. The Noni formulation of claim 9, wherein the amount of proxeronine is from about 0.1% to about 50% w/w of the Noni extract.

11. The Noni formulation of claim 9, wherein the amount of proxeronine is about 5% w/w of the Noni extract.

12. The Noni formulation of claim 1, wherein the chew base includes an effective amount of an invert sugar.

13. The Noni formulation of claim 12, wherein the amount of invert sugar is from about 1% to about 20% w/w of the formulation.

13. The Noni formulation of claim 13, wherein the amount of invert sugar is about 3% to 15% w/w of the formulation.

14. The Noni formulation of claim 12, wherein the invert sugar comprises a mixture of dextrose and fructose.

15. The Noni formulation of claim 14, wherein the dextrose and the fructose are each present in an amount of about 50% w/w of the invert sugar.

16. The Noni formulation of claim 12, wherein the invert sugar is provided by rice syrup and includes a mixture of glucose and maltose.

17. The Noni formulation of claim 16, wherein the amount of rice syrup is from about 15% to about 40% w/w of the formulation.

18. A method of making a Noni extract formulation comprising the steps of:
a) preparing a chew base containing an effective amount of an invert sugar in a conventional manner in a heated liquid phase form;
b) partially cooling said heated liquid phase to a temperature at which Noni extract is stable;
c) adding a desired amount of Noni extract to said partially cooled liquid phase;
d) agitating said partially cooled liquid phase until the Noni extract is substantially uniformly dispersed therein; and
e) further cooling said partially cooled liquid phase to a solid thereby resulting in said Noni extract formulation.

19. The method of claim 18, wherein the temperature of said partially cooled liquid caramel phase is between about 160° F. to about 220° F. at the time said Noni extract is added.

20. The method of claim 19, wherein the temperature is from about 180° F. to 200° F.

21. The method of claim 18, wherein the amount of Noni extract is from about 0.1% to 24% w/w of the Noni formulation.

22. The method of claim 18, wherein the amount of Noni extract is from about 9% to 13% w/w of the Noni formulation.

23. The method of claim 18, wherein the invert sugar is provided by an effective amount of rice syrup.

24. The method of claim 18, further comprising the step of:
dividing the Noni extract formulation into individual serving size portions.

25. A method of increasing the efficacy of a Noni extract dose comprising the steps of:
a) distributing an amount of Noni extract into a chew composition containing an insulin level enhancing amount of sugar and an invert sugar; and
b) orally administering the composition.

26. The method of claim 25, wherein the amount of invert sugar is from about 1% to about 20% w/w of the formulation.

27. The method of claim 25, wherein the amount of invert sugar is about 3% to 15% w/w of the formulation.

28. The method of claim 25, wherein the invert sugar comprises a mixture of dextrose and fructose.

29. The method of claim 25, wherein the dextrose and the fructose are each present in an amount of about 50% w/w of the invert sugar.

30. The method of claim 25, wherein the invert sugar is provided by rice syrup and includes a mixture of glucose and maltose.

31. The method of claim 30, wherein the amount of rice syrup is from about 15% to about 40% w/w of the formulation.

32. The method of claim 25, wherein the step of administering the composition is performed at a time when digestive juices in the upper gastrointestinal tract are at a minimum.

33. The method of claim 25, wherein the chew composition is administered in a total dosage that is insufficient to increase upper gastro intestinal tract digestive action to a level which substantially inactivates one or more active agents in the Noni extract.

34. A method of reducing or eliminating an undesirable taste caused by Noni extract comprising the step of:
a) distributing the Noni extract into a chew composition containing a sufficient amount of invert sugar to reduce or eliminate any disagreeable taste caused by the Noni extract.

35. The method of claim 34, wherein the amount of invert sugar is from about 1% to about 20% w/w of the formulation.

36. The method of claim 34, wherein the amount of invert sugar is about 3% to 15% w/w of the formulation.

37. The method of claim 34, wherein the invert sugar comprises a mixture of dextrose and fructose.

38. The method of claim 37, wherein the dextrose and the fructose are each present in an amount of about 50% w/w of the invert sugar.

39. The method of claim 34, wherein the invert sugar is provided by rice syrup and includes a mixture of glucose and maltose.

40. The method of claim 39, wherein the amount of rice syrup is from about 15% to about 40% w/w of the formulation.

41. A Noni extract formulation comprising a chew base having an insulin level enhancing amount of sugar and invert sugar, and a therapeutically effective amount of a Noni extract, wherein said invert sugar is present in an amount sufficient to reduce a disagreeable taste imparted by the Noni extract, and the formulation has a total dosage size that is insufficient to increase upper gastro intestinal tract digestive activity to a degree which substantially inactivates one or more active agents in the Noni extract.

42. A Noni extract formulation comprising a chew base having an insulin level enhancing amount of sugar and invert sugar, and a therapeutically effective amount of a Noni extract.
